(19) United States
(12) Patent Application Publication
(10) Pub. No.: US 2020/0260921 A1
(43) Pub. Date: Aug. 20, 2020

(54) HANDHELD DEVICE FOR DRYING THE LOWER EXTREMITIES

(71) Applicant: Natalie Delores Phillips, Chesapeake, VA (US)

(72) Inventor: Natalie Delores Phillips, Chesapeake, VA (US)

(21) Appl. No.: 16/456,791

(22) Filed: Jun. 28, 2019

Related U.S. Application Data
(60) Provisional application No. 62/918,904, filed on Feb. 19, 2019.

(51) Int. Cl. A47K 10/00; A61F 4/00

(52) U.S. Cl. CPC A47K 10/00; A61F 4/00

(57) ABSTRACT

A handheld device for assisting in the self-drying of the extremities of a human user is provided. The handheld device provides toe and foot drying components which are removably attachable to opposing ends of an elongated handle so that a user with medical disabilities or physical deficits can dry their lower extremities. Each drying component may be covered in an absorbent material. A distal end of toe dryer component may be dimensioned and adapted to slide between the toes of the user, while the foot dryer component is adapted to dry flatter surfaces along the feet, back and other portions of the human body, as well as supporting surfaces such as the floor or seat found in and around bathing facilities.

### CROSS-REFERENCE TO RELATED APPLICATION

This application claims the benefit of priority of U.S. provisional application No. 62/918,904, filed 19 Feb. 2019 the contents of which are herein incorporated by reference.

### BACKGROUND OF THE INVENTION

The present invention relates to handheld devices for drying the lower extremities and, more particularly, to a long-handled adaptive device used to dry the feet and between toes after shower, bathing, or swimming. The present invention is adapted to help improve and facilitate the independence of user's with medical disabilities and/or physical deficits, such as the elderly, during self-caring activities typical of daily living. The present invention can also help skin integrity as well as prevent falls.

When drying one's lower extremities, such as the feet and between the toes, the required flexibility and balance can be a challenge for those without physical disabilities. For those with medical disabilities and/or physical deficits such demands can be prohibitive. As a result, currently individuals with limited reach, flexibility or balance need to depend on someone else to thoroughly perform drying of his/her feet and toes.

As can be seen, there is a need for a long-handled adaptive device used to dry a user's lower extremities. The present invention solves an individual's inability to reach their lower extremities due to medical disabilities and/or physical disabilities deficits that causes limitations. Through increasing one's ability to dry between the toes using the long-handled adaptive device without bending over or bending down, the present invention promotes an individual's self-care and thus independence. Additionally, the present invention is adapted to promote skin integrity and foot care which is particularly important for a diabetic. Furthermore, the present invention can help decrease falls when used as either an intervention through keeping feet dry prior to or immediately upon exiting the shower, or through being used as a tool to keep supporting surfaces dry.

### SUMMARY OF THE INVENTION

In one aspect of the present invention, a handheld device for drying the lower extremities includes the following: an elongate handle extending between a first end and an opposing second end; a toe dryer provided along the first end; and the toe dryer having a conical shape so that a distal end thereof is dimensioned and adapted to slide between toes of a human user.

In another aspect of the present invention, the handheld device for drying the lower extremities includes the following: an elongate handle extending between a first end and an opposing second end; a toe dryer provided along the first end; and the toe dryer having a conical shape so that a distal end thereof is dimensioned and adapted to slide between toes of a human user; an absorbent material covering an external portion of the toe dryer; the first end providing a protrusion having an external threading; a cavity provided in a proximal end of the toe dryer; and the cavity providing an internal threading for operatively associating with the external threading so that the toe dryer is removable from the first end. And in further embodiments, provide a foot dryer provided along the second end, the foot dryer having a rectangular shape; a foot absorbent material covers an external portion of the foot dryer; the second end providing a foot protrusion having an external threading; a foot cavity provided in a proximal end of the foot dryer; and the foot cavity providing an internal threading for operatively associating with the external threading of the foot protrusion so that the foot dryer is removable from the second end.

In another aspect of the present invention, a method for an individual with medical disabilities or physical deficits to dry between their toes, includes the following: providing the handheld device for drying the lower extremities disclosed above; sliding the distal end of the toe dryer between two toes of said individual; and rotating the elongated handle until the absorbent material has dried the space between the two toes.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, demonstrating a toe dryer component;

FIG. 2 is a perspective view of an exemplary embodiment of the present invention, demonstrating a foot dryer component;

FIG. 3 is an exploded elevation view of an exemplary embodiment of the present invention;

FIG. 4 is a detailed side section view of an exemplary embodiment of the present invention;

FIG. 5 is an elevation view of an exemplary embodiment of a foot drying configuration of the present invention; and

FIG. 6 is an elevation view of an exemplary embodiment of a toe drying configuration of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a handheld device for assisting in the self-drying of the extremities of a human user. The handheld device provides toe and foot drying components which are removably attachable to opposing ends of an elongated handle so that a user with medical disabilities or physical deficits can dry their lower extremities. Each drying component may be covered in an absorbent material. A distal end of toe dryer component may be dimensioned and adapted to slide between the toes of the user, while the foot dryer component is adapted to dry flatter surfaces along the feet, back and other portions of the human body, as well as supporting surfaces such as the floor or seat found in and around bathing facilities.

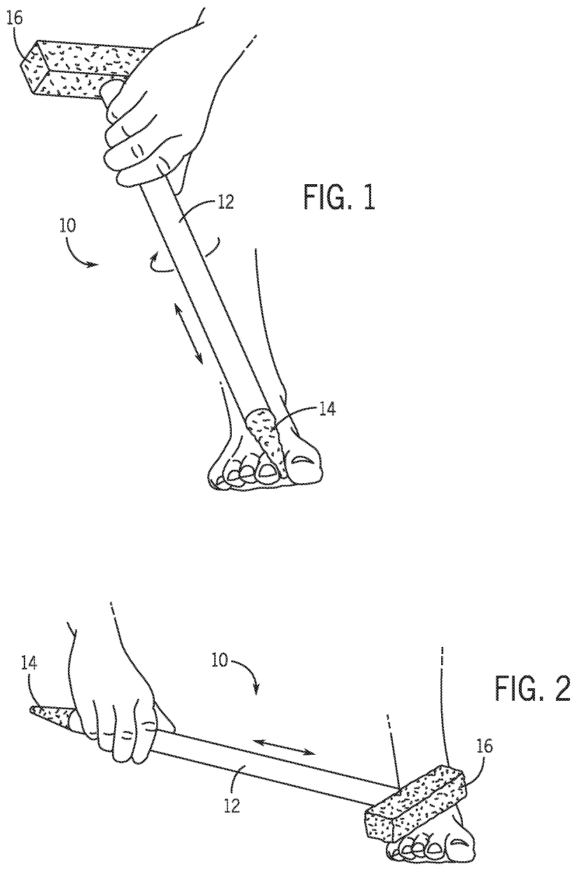

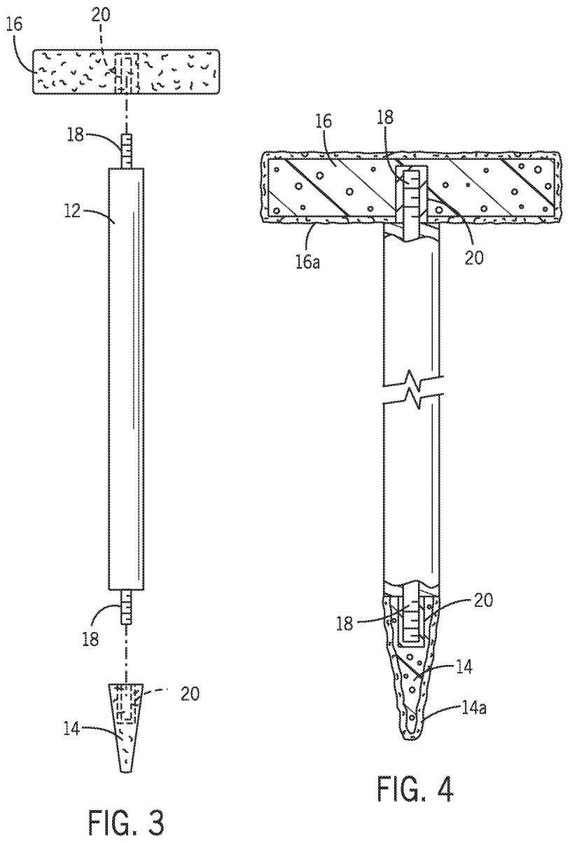

Referring to FIGS. 1 through 6, the present invention may include a handheld device 10 for drying the lower extremities. The handheld device 10 may provide an elongated handle 12 extending between a first end and a second end.

The first end may provide a toe dryer 14 tapering to a conical shape at a distal end thereof. The second end may provide a foot dryer 16 having a rectangular shape, offering a plurality of surface to absorb water, possible from different sources. It should be understood that the shapes of the toe dryer 14 and the foot dryer 16 may vary relative to those shown in the appended Figures as long as they function in accordance with the present invention as disclosure herein.

The elongated handle 12 may be made of wood, plastic, or other suitable material and extend between 24 to 36 inches in length and have a diameter between one and three inches. The first and second ends may provide protrusions with external threading 18 for removably attaching to the toe dryer 14 and the foot dryer 16, respectively. And so, the toe dryer 14 and the foot dryer 16 providing cavities with internal threading 20 for operatively receiving and engaging the protrusions and external threading 18, respectively.

The toe dryer 14 and the foot dryer 16 may include external and/or be covered with spongy, absorption material 14a and 16a, respectively. Such spongy material 14a/16a may be terrycloth or the like. The distal end of the toe dryer 14 is dimensioned and adapted to slide between the toes of a human user.

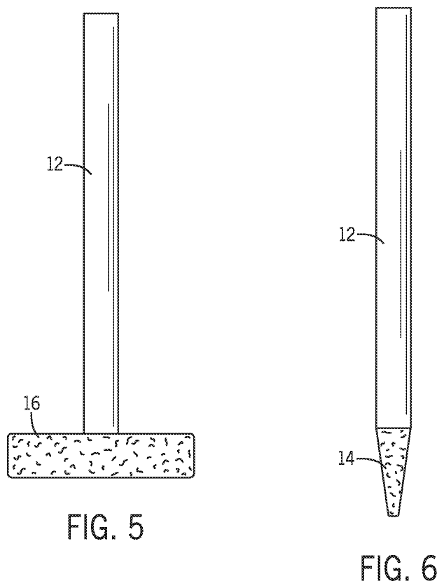

It being understood that in certain embodiments, the toe dryer 14 attachment may be switched out with the foot dryer 16 attachment during use, as illustrated in FIGS. 5 and 6, instead of having a two-headed device.

A method of using the present invention may include the following. The handheld device 10 for drying the lower extremities disclosed above may be provided. A user may attach the toe dryer 14 and the foot dryer 16 on opposing ends of the elongated handle 12, respectively. Then the user may slide the distal end of the toe dryer 14 between the toes and rotate the elongated handle 12 until the toe area is dry, preventing skin a breakdown between toes (especially important for diabetics). Then, the user may utilize the foot dryer 16 to dry their feet as well as to dry the floor of water, for example after a shower, so as to help reduce slips and falls after showering. The toe and foot dryers 14 and 16 may also be used on other hard-to-reach parts of the user's anatomy, such as along their back, to dry/absorb water after showering.

It should be understood, of course, that the foregoing relates to exemplary embodiments of the invention and that modifications may be made without departing from the spirit and scope of the invention as set forth in the following claims.

What is claimed is:

1. A handheld device for drying the lower extremities, comprising:
an elongate handle extending between a first end and an opposing second end;
a toe dryer provided along the first end; and
the toe dryer having a conical shape so that a distal end thereof is dimensioned and adapted to slide between toes of a human user.

2. The handheld device for drying the lower extremities of claim 1, further comprising:
a foot dryer provided along the second end, the foot dryer having a rectangular shape.

3. The handheld device for drying the lower extremities of claim 1, further comprising: an absorbent material covering an external portion of the toe dryer.

4. The handheld device for drying the lower extremities of claim 1, further comprising: a protrusion having an external threading; the protrusion extending from the first end; a cavity provided in a proximal end of the toe dryer; the cavity providing an internal threading for operatively associating with the external threading so that the toe dryer is removable from the first end.

5. A handheld device for drying the lower extremities, comprising:
an elongate handle extending between a first end and an opposing second end;
a toe dryer provided along the first end; and
the toe dryer having a conical shape so that a distal end thereof is dimensioned and adapted to slide between toes of a human user;
an absorbent material covering an external portion of the toe dryer;
the first end providing a protrusion having an external threading;
a cavity provided in a proximal end of the toe dryer; and
the cavity providing an internal threading for operatively associating with the external threading so that the toe dryer is removable from the first end.

6. The handheld device for drying the lower extremities of claim 5, further comprising:
a foot dryer provided along the second end, the foot dryer having a rectangular shape.

7. The handheld device for drying the lower extremities of claim 6, further comprising:
a foot absorbent material covers an external portion of the foot dryer;
the second end providing a foot protrusion having an external threading;
a foot cavity provided in a proximal end of the foot dryer; and
the foot cavity providing an internal threading for operatively associating with the external threading of the foot protrusion so that the foot dryer is removable from the second end.

8. The handheld device for drying the lower extremities of claim 7, wherein the elongated handle extends for approximately 24 inches.

9. A method for an individual with medical disabilities or physical deficits to dry between their toes, comprising:
providing the handheld device for drying the lower extremities of claim 5;
sliding the distal end of the toe dryer between two toes of said individual; and
rotating the elongated handle until the absorbent material has dried the space between the two toes.
